(19) United States
(12) Patent Application Publication
(10) Pub. No.: US 2012/0045786 A1
(43) Pub. Date: Feb. 23, 2012

(54) OPTO-FLUIDIC MICROSCOPE DIAGNOSTIC SYSTEM

(76) Inventor: Curtis W. Stith, Santa Cruz, CA (US)

(21) Appl. No.: 13/114,990

(22) Filed: May 24, 2011

Related U.S. Application Data
(60) Provisional application No. 61/453,100, filed on Mar. 15, 2011.
(60) Provisional application No. 61/375,227, filed on Aug. 19, 2010.

(51) Int. Cl. C12Q 1/02; C12M 1/34

(52) U.S. Cl. 435/29; 435/287.2; 435/288.7

(57) ABSTRACT

An image sensor integrated circuit may contain image sensor pixels. Channels containing a fluid with cells or other material may be formed on top of the image sensor. The image sensor pixels may form imagers. Each imager may be located in a respective one of the channels. Reactant chambers may be used to expose the particles in the fluid to reactant. The imagers may gather images of the cells or other particles as the fluid passes over the imagers following exposure to the reactant. Spent sample chambers at the ends of the channels may be used to collect the fluid after the fluid has passed over the imagers. Image data from the imagers may be processed by control circuitry on the image sensor integrated circuit and external equipment.

This application claims the benefit of provisional patent application No. 61/453,100, filed Mar. 15, 2011 and provisional patent No. 61/375,227, filed Aug. 19, 2010, which are hereby incorporated by reference herein in their entireties.

### BACKGROUND

This relates generally to systems such as opto-fluidic microscope systems, and, more particularly, to using such systems to image fluid samples containing cells and other specimens.

Opto-fluidic microscopes have been developed that can be used to generate images of cells and other biological specimens. The cells are suspended in a fluid. The fluid flows over a set of image sensor pixels in a channel. The image sensor pixels may be associated with an image sensor pixel array that is masked using a metal layer with a pattern of small holes. In a typical arrangement, the holes and corresponding image sensor pixels are arranged in a diagonal line that crosses the channel. As cells flow through the channel, image data from the pixels may be acquired and processed to form high-resolution images of the cells.

### BRIEF DESCRIPTION OF THE DRAWINGS

FIG. 1 is a cross-sectional diagram of an illustrative system for imaging cells and other biological specimens in accordance with an embodiment of the present invention.

FIG. 2 is a cross-sectional side view of a portion of an image sensor pixel array of the type that may be used in a fluid channel in a system of the type shown in FIG. 1 in accordance with an embodiment of the present invention.

FIG. 3 is a top view of an illustrative fluid channel having image pixels arranged in a line to form an imager in accordance with an embodiment of the present invention.

FIG. 4 is a top view of an illustrative fluid channel that contains a gate structure for controlling the flow of fluid in accordance with an embodiment of the present invention.

FIG. 5 is a top view of an illustrative system having multiple channels with multiple imagers in accordance with an embodiment of the present invention.

FIG. 6 is a graph of illustrative control signals that may be applied to the gate structures in respective channels to ensure that a sample is exposed to different reactants for appropriate amounts of time before being imaged by respective imagers in accordance with an embodiment of the present invention.

FIG. 7 is a perspective view of illustrative system environment in which an opto-fluidic microscope imaging system of the type shown in FIG. 1 may be used to gather image data of cells and other biological specimens in accordance with an embodiment of the present invention.

FIG. 8 is a flow chart of illustrative steps involved in using a system with fluid channels and imagers to evaluate samples in accordance with an embodiment of the present invention.

### DETAILED DESCRIPTION

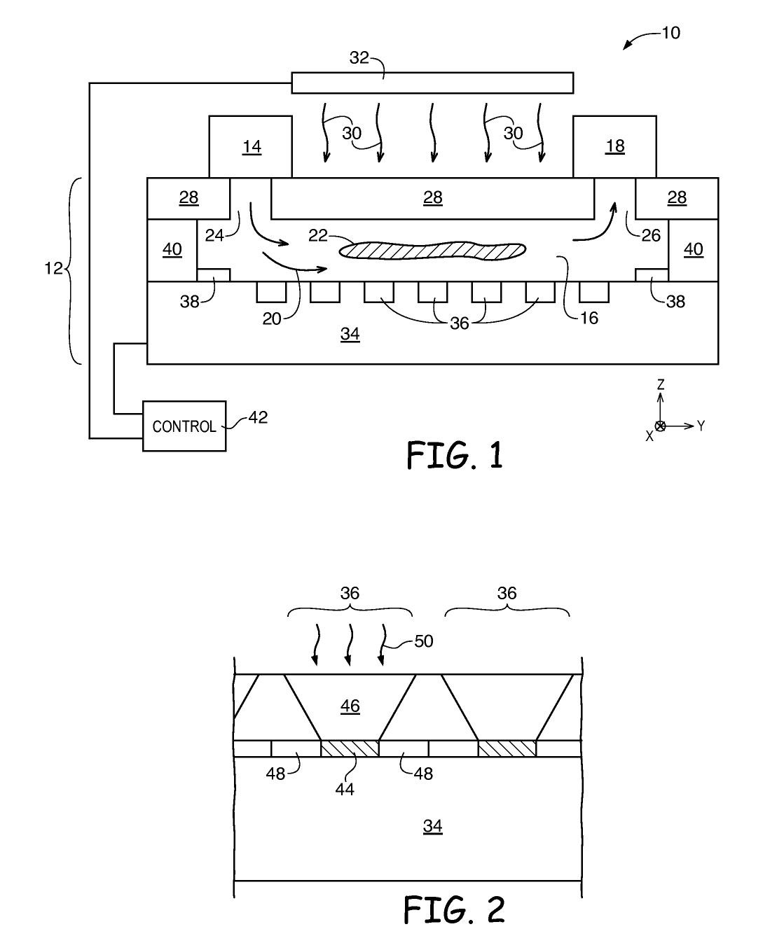

An opto-fluidic microscope system of the type that may be used to image and otherwise evaluate cells and other samples such as biological specimens is shown in FIG. 1. As shown in FIG. 1, system 10 may include opto-fluidic microscope 12. Microscope 12 may include an image sensor integrated circuit such as image sensor integrated circuit 34. Image sensor integrated circuit 34 may be formed from a semiconductor substrate material such as silicon and may contain numerous image sensor pixels 36. Complementary metal-oxide-semiconductor (CMOS) technology or other image sensor integrated circuit technologies may be used in forming image sensor pixels 36 and integrated circuit 34.

Image sensor pixels 36 may form part of an array of image sensor pixels on image sensor integrated circuit 34 (e.g., a rectangular array). Some of the pixels may be actively used for gathering light. Other pixels may be inactive or may be omitted from the array during fabrication. In arrays in which fabricated pixels are to remain inactive, the inactive pixels may be covered with metal or other opaque materials, may be depowered, or may otherwise be inactivated. There may be any suitable number of pixels fabricated in integrated circuit 34 (e.g., tens, hundreds, thousands, millions, etc.). The number of active pixels in integrated circuit 34 may be tens, hundreds, thousands, or more).

Image sensor integrated circuit 34 may be covered with a transparent layer of material such as glass layer 28 or other covering layers. Layer 28 may, if desired, be colored or covered with filter coatings (e.g., coatings of one or more different colors to filter light). Structures such as standoffs 40 (e.g., polymer standoffs) may be used to elevate the lower surface of glass layer 28 from the upper surface of image sensor integrated circuit 34. This forms one or more channels such as channels 16. Channels 16 may have lateral dimensions (dimensions parallel to dimensions x and z in the example of FIG. 1) of a millimeter or less (as an example). The length of each channel (the dimension of channel 16 along dimension y in the example of FIG. 1) may be 1-10 mm, less than 10 mm, more than 10 mm, or other suitable length. Standoff structures 40 may be patterned to form sidewalls for channels such as channel 16.

During operation, fluid flows through channel 16 as illustrated by arrows 20. A fluid source such as source 14 may be used to introduce fluid into channel 16 through entrance port 24. Fluid may, for example, be dispensed from a pipette, from a drop on top of port 24, from a fluid-filled reservoir, from tubing that is coupled to an external pump, etc. Fluid may exit channel 16 through exit port 26 and may, if desired, be collected in reservoir 18. Reservoirs (sometimes referred to as chambers) may also be formed within portions of channel 16.

The rate at which fluid flows through channel 16 may be controlled using fluid flow rate control structures. Examples of fluid flow rate control structures that may be used in system 10 include pumps, electrodes, microelectromechanical systems (MEMS) devices, etc. If desired, structures such as these (e.g., MEMs structures or patterns of electrodes) may be used to form fluid flow control gates (i.e., structures that selectively block fluid flow or allow fluid to pass and/or that route fluid flow in particular directions). In the example of FIG. 1, channel 16 has been provided with electrodes such as electrodes 38. By controlling the voltage applied across electrodes such as electrodes 38, the flow rate of fluids in channel 16 such as ionic fluids may be controlled by control circuitry 42.

Fluid 20 may contain cells such as cell 22 or other biological elements or particles. As cells such as cells 22 pass by sensor pixels 36, image data may be acquired. In effect, the cell is “scanned” across the pattern of sensor pixels 36 in channel 16 in much the same way that a printed image is scanned in a fax machine. Control circuitry 42 (which may be implemented as external circuitry or as circuitry that is embedded within image sensor integrated circuit 34) may be used to process the image data that is acquired using sensor pixels 36. Because the size of each image sensor pixel 36 is typically small (e.g., on the order of 0.5-3 microns or less in width), precise image data may be acquired. This allows high-resolution images of cells such as cell 22 to be produced. A typical cell may have dimensions on the order of 1-10 microns (as an example). Images of other samples (e.g., other biological specimen or other particles) may also be acquired in this way. Arrangements in which cells are imaged are sometimes described herein as an example.

During imaging operations, control circuit 42 (e.g., on-chip and/or off-chip control circuitry) may be used to control the operation of light source 32. Light source 32 may be based on one or more lamps, light-emitting diodes, lasers, or other sources of light. Light source 32 may be a white light source or may contain one or more light-generating elements that emit different colors of light. For example, light-source 32 may contain multiple light-emitting diodes of different colors or may contain white-light light-emitting diodes or other white light sources that are provided with different respective colored filters. If desired, layer 28 may be implemented using colored transparent material in one or more regions that serve as one or more color filters. In response to control signals from control circuitry 42, light source 32 may produce light 30 of a desired color and intensity. Light 30 may pass through glass layer 28 to illuminate the sample in channel 16.

A cross-sectional side view of illustrative image sensor pixels 36 is shown in FIG. 2. As shown in FIG. 2, image sensor pixels 36 on integrated circuit 34 may each include a corresponding photosensitive element such as photodiode 44. Light guides such as light guide 46 may be used to concentrate incoming image light 50 into respective photodiodes 44. Photodiodes 44 may each convert incoming light into corresponding electrical charge. Circuitry 48, which may form part of control circuitry 42 of FIG. 1, may be used to convert the charge from photodiodes 44 into analog and/or digital image data. In a typical arrangement, data is acquired in frames. Control circuitry 42 may convert raw digital data from one or more acquired image data frames into images of cells 22.

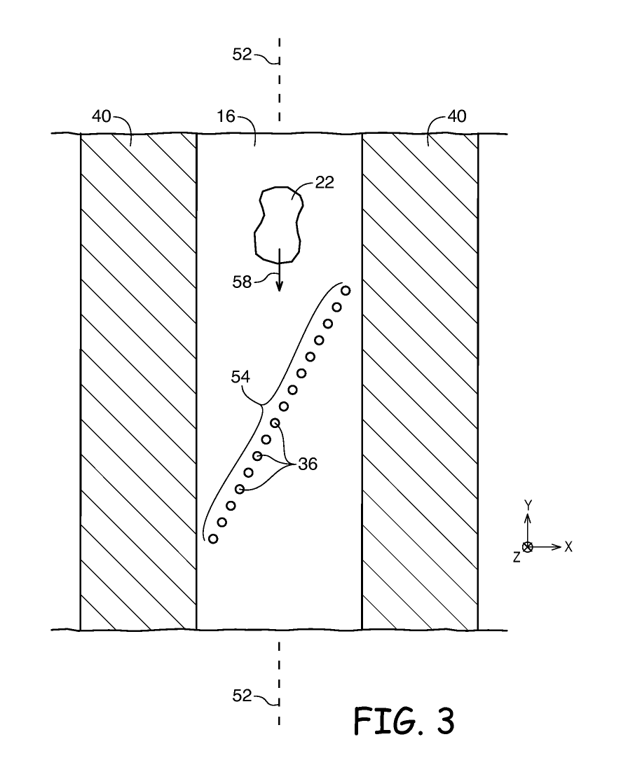

As shown in FIG. 3, pixels 36 in channel 16 may be arranged to form imager 54. Pixels 36 may be arranged in a diagonal line that extends across the width of channel 16 or may be arranged in other suitable patterns. The use of a diagonal set of image acquisition pixels 36 in channel 16 may help improve resolution (i.e., lateral resolution in dimension x perpendicular to longitudinal axis 52) by increasing the number of pixels 36 per unit length in dimension x. The image acquisition pixels 36 in channel 16 (i.e., the imager sensor pixels) are sometimes referred to as forming an image acquisition region, image sensor, or imager.

Light source 32 may be adjusted to produce one or more different colors of light during image acquisition operations. Channels 16 in system 10 may be provided with one or more imagers 54. The different colors of light may be used in gathering image data in different color channels. If desired, a different respective light color may be used in illuminating cells 22 as cells 22 pass each respective imager within a set of multiple imagers 54 in a given channel by moving in direction 58 with the fluid in the channel.

In some situations, it may be desirable to mix fluid 20 and/or cells 22 with a reactant. Examples of reactants that may be introduced into channel 16 with fluid 20 and cells 22 include diluents (e.g., fluids such as ionic fluids), dyes (e.g., fluorescent dyes) or other chemical compounds, biological agents such as antigens, antibodies (e.g., antibodies with dye), reagents, phosphors, electrolytes, analyte-specific antibodies, etc.

With one suitable arrangement, one or more reactants may be introduced within a portion of channel 16. The portion of channel 16 that receives the reactant may be, for example, a portion of channel 16 that has been widened or a portion of channel 16 that has the same width as the rest of the channel. Portions of channel 16 (whether widened or having other shapes) that receive reactant or that may be used to introduce sample material into channel 16 are sometimes referred to herein as chambers and reservoirs.

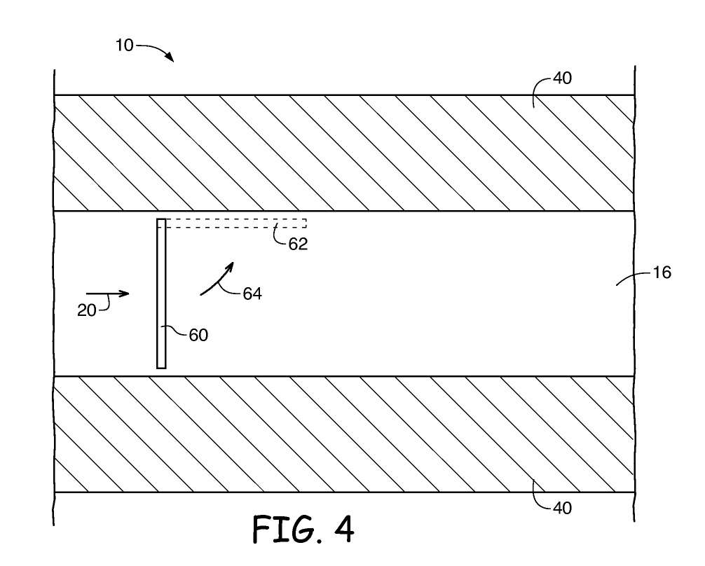

FIG. 4 shows how channels in system 10 may be provided with configurable gate structures (gating structures) such as gate structure 60. Gate structures such as gate structure 60 may have open and closed positions. In the example of FIG. 4, gate structure 60 in its closed position in which the flow of fluid 20 is blocked. When moved in direction 64 to open position 62 or when otherwise opened, gate structure 60 permits fluid 20 to flow through channel 16. Gate structures such as gate structure 60 may, for example, be formed from MEMs structures, electrode-based structures, or other structures that can selectively permit fluid to flow or block fluid from flowing. Electrodes such as electrodes 38 of FIG. 1 or other fluid control mechanisms (e.g., MEMs structures, external pumps, etc.) may be used to cause the sample fluid to flow through channel 16. Gate structures such as gate structure 60 may be used to selectively block the flow of the sample. For example, gate structure 60 may be placed in a closed position to momentarily prevent fluid from flowing and thereby ensure that the fluid remains in contact with a reactant for an amount of time that is appropriate for that reactant to interact with the sample. Once the appropriate amount of time has elapsed, control circuitry 42 may open gate structure 60 to allow the fluid sample to proceed past one or more imagers.

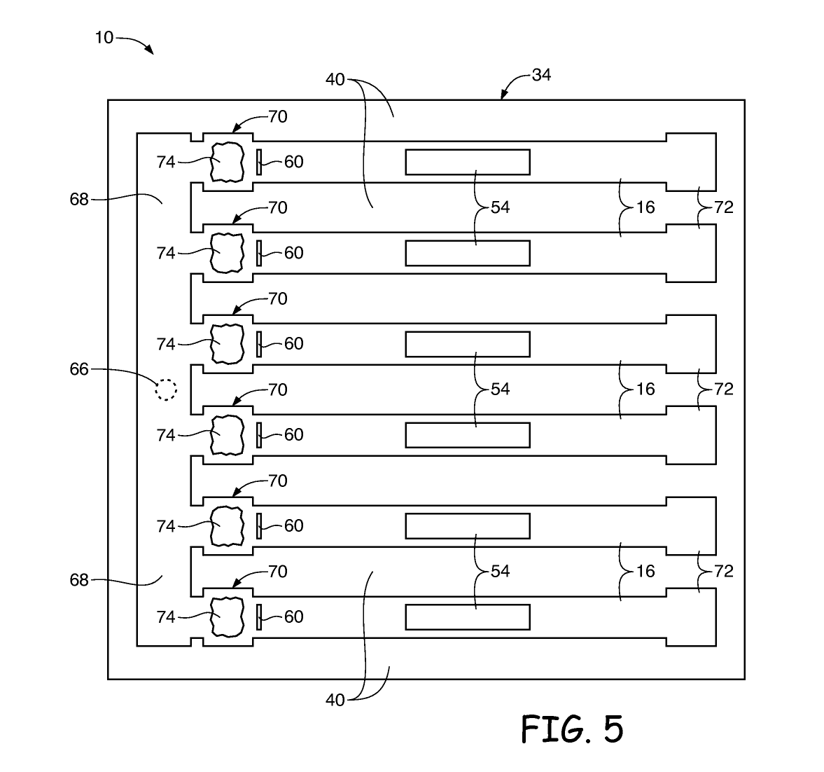

As shown in FIG. 5, system 10 may be formed on an image sensor integrated circuit substrate (substrate 34) that has multiple channels 16. Channels 16 may, in general, be arranged on the surface of substrate 34 in a pattern with parallel channel segments (as shown in FIG. 5), in a pattern with perpendicular channel segments, in a pattern in which channels branch from one another at non-parallel and non-perpendicular angles, or other suitable channel patterns. The arrangement of FIG. 5 is merely illustrative.

Sample reservoir 68 may have exit ports coupled to each of the channels. In the example of FIG. 5, there are six parallel channels 16, so there are six corresponding exit ports that couple sample reservoir 68 to channels 16. In systems with different numbers of channels (e.g., more than six channels or fewer than six channels), different corresponding numbers of exit ports may be formed in sample reservoir 68.

Fluid samples may be introduced into sample reservoir 68 through entrance port 66 (e.g., a hole in a cover such as hole 24 in cover layer 28 of FIG. 1). By introducing fluid into reservoir 68 through entrance port 66, a fluid sample may be distributed among the channels.

It may be desirable to introduce reactant into channels 16. For example, reactants may be used to make cells and other particles more visible within channels 16 (e.g., by staining the cells with dye, etc.). As shown in FIG. 5, reactant 74 may be supplied to each channel 16 using a corresponding reactant chamber 70. There may be one or more different reactants in each reactant chamber 70.

Gate structures 60 may be used to control the amount of time that the sample spends in each reactant chamber 70. In some situations (e.g., when a reactant is slow-acting or when a longer reactant exposure time is desired), it may be desirable to hold the sample in a particular reactant chamber for a relatively long period of time. In other situations (e.g., when a reactant is fast acting or when a shorter reactant exposure time is desired), it may be desirable to hold the sample in a reactant chamber for a relatively short period of time. Using gate structures 60 of FIG. 5, some portions of a sample may be exposed to reactant 74 for longer than others. Different reactants may also be placed in different respective chambers 70.

Consider, as an example, a situation in which a particular type of cell is to be imaged following staining of the cell with a dye. The appearance of the stained cell may be different depending on how long the cell is exposed to the reactant. It may therefore be desirable to expose some portions of the sample to the reactant for short periods of time, while exposing other portions of the same sample to the reactant for longer periods of time. The cell may also respond differently to different concentrations of the reactant and different types of reactants. Using reservoir 68, a sample may be distributed to each of the reactant chambers 70 in system 10. Reactant chambers 70 may hold one or more types of reactant 74 in one or more different concentrations. Gate structures 60 may be used to hold the sample in different reactant chambers for different amounts of time (i.e., different sample hold times).

Once the sample has been held in a reactant chamber for a sufficiently long period of time, the gate structure that is associated with that reactant chamber may be opened to release the sample into an adjoining channel. Upon release, the sample in each channel will flow past the imager 54 (or imagers) in that channel. The imager may be used in gathering image data for the sample. The image data may be processed to form images of the sample. The images that are formed may be displayed for a user on a monitor. Because each imager 54 can gather image data from a sample that has been exposed to reactant in a different way (e.g., a different reactant type, different exposure time, different reactant concentration, etc.), each imager 54 can gather a different type of image data. During image processing operations, the image data may be processed to form images of cells and other particles in the sample.

As shown in FIG. 5, after a portion of the sample passes by each imager 54, that portion of the sample may flow into a corresponding chamber 72. Chambers 72 may be spent sample reservoirs or may contain components for evaluating the sample. For example, chambers 72 may include image pixels that have been configured to serve as light sensors, light sources for illuminating the sample (e.g., for fluorescence measurements), heaters for heating the samples, additional reactant, etc.

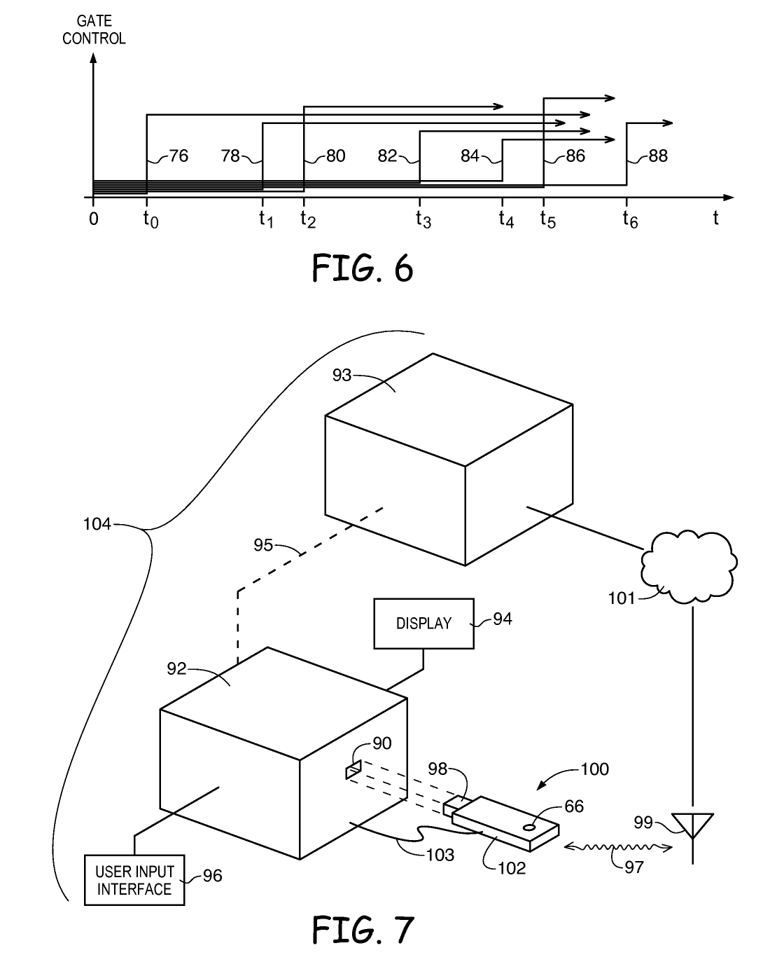

FIG. 6 is a graph showing how the control signals that are applied to each gate structure 60 in FIG. 5 may potentially be different. Each trace in the example of FIG. 6 corresponds to an illustrative control signal for a different respective one of the six gate structures 60 in FIG. 5. In this example, the status of the gate structures is controlled by the state of the control signal. When the control signal for a given gate structure is deasserted (e.g., when the control signal is taken low), the gate structure is held in its closed state. When the control signal for a given gate structure is asserted (e.g., when the control signal is taken high), the gate structure is placed in its open state. As shown in FIG. 6, at time t0, a first of the gate structures 60 (i.e., the uppermost gate structure 60 in FIG. 5) may be opened, whereas the remaining gate structures 60 remain closed. At time t1, a second of the gate structures 60 is opened by asserting control signal 78. The four remaining gate structures are likewise moved from their closed to open states at times t2, t3, t4, t5, and t6, respectively, as illustrated by control signals 80, 82, 84, 86, and 88. Using this type of arrangement, the portion of the fluid sample that is contained in the first reactant chamber (i.e., the sample in the uppermost reactant chamber in the example of FIG. 5) is exposed to a first reactant in a first concentration for a first period of time (i.e., time t0, assuming that the fluid is placed in the reactant chambers at time t=0). The portion of the fluid sample that is placed in the other reactant chambers is exposed to reactant for different exposure times (i.e., sample hold times t1, t2, t3, t4, t5, and t6). Each reactant chamber potentially has a different type of reactant and a different reactant concentration. The use of potentially different respective hold times for the sample in each reactant chamber allows the hold times for holding the sample in the reactant chambers to be individualized to the type and concentration of reactant in each reactant chamber and other factors.

FIG. 7 is a perspective view showing how an opto-fluidic microscope diagnostic system 100 may be configured to communicate with data analysis equipment 104. Data analysis equipment 104 may be based on one or more computers or other computing equipment. Equipment 104 may, for example, include computing equipment such as computing equipment 92. An associated display such as display 94 may be used in presenting visual information to a user such as images of cells and other samples acquired using system 100. User input interface 96 may be used to gather input from a user and to supply output for a user. For example, user input interface 96 may contain user input devices such as keyboards, keypads, mice, trackballs, track pads, etc. User input interface 96 may also include equipment for supplying output such as speakers for providing audio output, status indicator lights for providing visible output, etc.

Equipment 104 may include a data port such as data port 90. Data port 90 may be, for example, a Universal Serial Bus (USB) port. As shown in FIG. 7, system 100 may have a connector such as connector 98 (e.g., a USB connector) that is configured to mate with the connector in port 90. Connector 98 may be mounted in housing 102 of system 100. System 100 may include a fluid sample entrance port such as port 66. Port 66 may be aligned with port 66 of FIG. 5, so that samples that are placed in port 66 of system 100 flow into sample reservoir 68 of microscope 12 within housing 102. After a sample has been introduced into system 100 through port 66, control circuitry 42 (FIG. 1) may be used to gather image data for forming one or more sample images.

After sample processing is complete, the user may insert system 100 into port 90, so that the data from system 100 may be passed to equipment 104 and further analyzed (e.g., to produce images of the sample from raw image data, to produce enhanced images, etc.). Alternatively, system 100 may be connected to computing equipment 92 via a wired connection such as wired connection 103. Computing equipment 92 may be a portable electronic device (e.g., a mobile phone, a personal digital assistant, laptop computer, or other computing equipment). Computing equipment 92 may be used to process data from system 100. Computing equipment 92 may be used to transmit data from system 100 to computing and data processing equipment 93 along communications path 95. Communications path 95 may be a wired or wireless connection. Communications path 95 may be used to directly transfer data from system 100 to computing and data analysis equipment 93 or may be used to transfer data from system 100 to computing and data analysis equipment 93 over a wired or wireless network. Computing and data processing equipment 93 may be a remote mainframe computer, may be a cloud computing network (i.e. a network of computers on which software can be run from computing equipment 92) or other computing equipment.

System 100 may have wireless transmitting circuitry configured to transfer data over wireless communication path 97 to antenna 99. Antenna 99 may relay data communicated wirelessly from system 100 to a network 101 and to computing and data processing equipment 93. Equipment such as opto-fluidic microscope system 100 may be produced inexpensively in volume and may be disposed of after a single use (as an example).

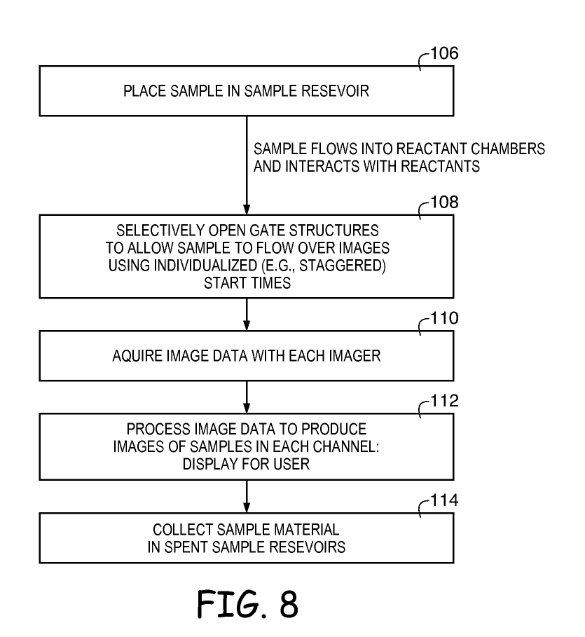

Illustrative steps involved in using an opto-fluidic microscope system to gather and analyze data on a sample are shown in FIG. 8. At step 106, a user of the system may place a sample in sample reservoir 68 (FIG. 5) through sample entrance port 66 (FIGS. 5 and 7). Once the sample flows into reservoir 68 and associated reactant chambers 70, the sample will interact with the reactant.

Different reactant chambers may require different amounts of sample hold time. Accordingly, control circuitry 42 may selectively activate gate structures 60 during the operations of step 108. Control circuitry 42 may, for example, open gate structures 60 in different channels at different times, as described in connection with the gate control signals of FIG. 6. This causes the sample fluid from each reactant chamber to flow over a corresponding imager after being held for a different respective sample hold time.

At step 110, as the sample fluid flows over imagers 54, imagers 54 acquire image data for the cells or other particles in the fluid.

Image processing operations may be performed in control circuitry 42 of system 100 and/or equipment 104 (FIG. 7) following transfer of image data from system 100 to equipment 104. In particular, at step 112, control circuitry associated with system 100 and/or equipment 104 may be used in processing the image data that was acquired during the operations of step 110 to form images of cells and other particles in the sample fluid of each channel. Because the sample was potentially exposed to different reactant environments in each reactant chamber, the images acquired by each of the imagers may provide complementary information about the sample.

Spent sample material may be collected in chambers 72 (FIG. 5). If desired, chambers 72 may be used to further analyze the sample material. For example, fluorescence measurements and other measurements may be made using light sources, light sensors, and other components associated with chambers 72.

Various embodiments have been described illustrating apparatus for imaging samples of fluids containing cells and other materials. An integrated circuit such as an image sensor array integrated circuit may be provided with fluid channels. Sets of image sensor pixels from an image sensor array on the integrated circuit may form imagers in the fluid channels. A sample may be introduced into a channel for imaging by the imagers. Chambers may be provided for adding dilutant and other reactants such as dyes, antigens, antibodies, chemical compounds, and other materials to the sample fluid. The channel structures on the integrated circuit may have multiple channels (branches). Gate structures such as microelectromechanical systems (MEMs) gate structures may be used to selectively route fluid through various channels from respective reactant chambers. Each reactant chamber may have a potentially different reactant and different concentration of reactant. Control circuitry may activate the gate structures to ensure that each portion of the sample spends an optimum amount of time in its reactant chamber before flowing over an imager in a corresponding channel.

The foregoing is merely illustrative of the principles of this invention which can be practiced in other embodiments.

What is claimed is:

1. Apparatus, comprising:
an image sensor integrated circuit containing image sensor pixels that form a plurality of imagers;
a plurality of reactant chambers containing reactant; and
a plurality of fluid channels on the image sensor integrated circuit that are each configured to receive fluid from a respective one of the reactant chambers.

2. The apparatus defined in claim 1 wherein the imagers are located within the fluid channels.

3. The apparatus defined in claim 2 wherein each fluid channel contains at least one of the imagers.

4. The apparatus defined in claim 3 further comprising gate structures that control fluid flow between the reactant chambers and the fluid channels.

5. The apparatus defined in claim 4 further comprising control circuitry that selectively opens and closes the gate structures to respectively permit the fluid to flow between the reactant chambers and the fluid channels and prevent the fluid from flowing between reactant chambers and the fluid chambers.

6. The apparatus defined in claim 5 wherein the reactant comprises dye.

7. The apparatus defined in claim 5 wherein the reactant comprises a reactant selected from the group consisting of: antigens, antibodies, phosphors, electrolytes, and analyte-specific antibodies.

8. The apparatus defined in claim 7 further comprising a sample port that is configured to receive the fluid, wherein the reactant chambers are configured to receive the fluid from the sample port.

9. The apparatus defined in claim 8 wherein the fluid comprises a sample containing cells.

10. The apparatus defined in claim 9 further comprising a plurality of spent sample chambers that receive the sample after the sample has passed over the imagers.

11. A method for analyzing fluid samples with an image sensor integrated circuit that has a plurality of image sensor pixels organized to form imagers in fluid channels on the image sensor integrated circuit, wherein the fluid channels include gate structures interposed between the fluid channels and respective reactant chambers, comprising:
selectively opening the gate structures to allow fluid to flow from the reactant chambers over the imagers in the channels.

12. The method defined claim 11 wherein selectively opening the gate structures comprises opening each of the gate structures at a different time.

13. The method defined in claim 12 further comprising:
gathering image data with the imagers as the fluid flows from the reactant chambers over the imagers.

14. The method defined in claim 13 wherein the fluid includes cells, the method further comprising:
exposing the cells to the reactant in the reactant chambers.

15. The method defined in claim 14 wherein gathering the image data comprises gathering image data on the cells that have been exposed to the reactant.

16. The method defined in claim 15 further comprising transferring the image data from the image sensor integrated circuit to computing equipment.

17. The method defined in claim 16 further comprising collecting at least some of the fluid that has flowed over the imagers in spent sample chambers coupled to the channels.

18. Apparatus, comprising:
an image sensor integrated circuit containing image sensor pixels;
a plurality of channels on the image sensor integrated circuit that are configured to receive a sample of fluid, wherein the image sensor pixels are configured to form a plurality of imagers, wherein each of the imagers is contained within a different respective one of the channels; and
a plurality of reactant chambers on the image sensor integrated circuit each of which is coupled to a respective one of the channels and each of which contains a reactant selected from the group consisting of: antigens, antibodies, phosphors, electrolytes, and analyte-specific antibodies.

19. The apparatus defined in claim 18 wherein the reactant chambers each contain dye, wherein the sample of fluid contains cells that are exposed to the dye in the reactant chambers, and wherein the imagers are configured to acquire image data as a the cells that have been exposed to the dye pass the imagers.

20. The apparatus defined in claim 19 further comprising a sample port that is configured to receive the sample of fluid and that is configured to distribute the sample of fluid to each of the plurality of reactant chambers, wherein the reactant in a first of the reactant chambers is different than the reactant in a second of the reactant chambers.
